(19) United States
(12) Patent Application Publication
(10) Pub. No.: US 2023/0312485 A1
(43) Pub. Date: Oct. 5, 2023

(54) A PRECIPITATION PROCESS FOR AMORPHOUS LETERMOVIR

(71) Applicant: Lupin Limited, Mumbai (IN)

(72) Inventors: Girij Pal Singh, Pune (IN); Radhakrishna Bhikaji Shivdavkar, Pune (IN); Govind Dnyanoba Ausekar, Pune (IN); Mithun Dasharath Surwase, Pune (IN); Rajendra Somnath Mhaske, Pune (IN); Suhas Ganpat Tambe, Pune (IN)

(21) Appl. No.: 18/021,760

(22) PCT Filed: Aug. 7, 2021
(86) PCT No.: PCT/IB2021/057299

§ 371 Date: Feb. 16, 2023

(30) Foreign Application Priority Data
Aug. 17, 2020 (IN) 202021035411

(51) Int. Cl. C07D 239/84

(52) U.S. Cl. CPC C07D 239/84

(57) ABSTRACT

A precipitation process for amorphous Letermovir, contains residual solvents in accordance with ICH guidelines and is suitable for the preparation of orally administered pharmaceutical formulations. The formulations of the amorphous Letermovir are intended for use in methods of prophylaxis or methods of treatment of viral diseases, in particular human cytomegalovirus (HCMV) infections.

### FIELD OF THE INVENTION

The present invention is related to a process of isolating amorphous Letermovir, contains residual solvents in accordance with ICH guidelines by using precipitation process. The present precipitation process makes amorphous Letermovir ready to be formulated in a solid pharmaceutical formulation for oral administration.

### BACKGROUND

Many pharmaceutical solids can exist in different physical forms. Polymorphism is often characterized as the ability of a drug substance to exist as two or more crystalline phases that have different arrangements and/or conformations of the molecules in the crystalline lattice. Amorphous solids consist of disordered arrangements of molecules and do not possess a distinguishable crystal lattice.

Polymorphs of a pharmaceutical solid may have different physical and solid-state chemical properties. These polymorphs differ in internal solid-state structure and, therefore, possess different chemical and physical properties, including packing, thermodynamic, spectroscopic, kinetic, and mechanical properties. These properties can have a direct impact on drug product quality/performance, including stability, dissolution, and bioavailability.

The most stable polymorphic form of a drug substance is often used in a pharmaceutical formulation because it has the lowest potential for conversion from one polymorphic form to another. On the other hand, metastable forms and even amorphous forms may be chosen to enhance the bioavailability of the drug product. Amorphous form, being a disorganized solid mass, does not need to lose crystal structure before dissolution in the gastric juices, and thus often has greater bioavailability than a crystalline form. Even if amorphous form is desirable for formulation, its preparation on industrial scale is often problematic. Many processes used to prepare amorphous form of an active pharmaceutical ingredient (API) are not suitable for industrial scale. Various processes for preparation of amorphous form are known in the art like solidification of melt, reduction of particle size, spray-drying, lyophilization, removal of a solvent from crystalline structure, evaporation of a solvent, precipitation of acids and bases by change in pH and others as techniques employed to obtain amorphous form of an API.

Many of these processes, however, are not practical on an industrial scale. For example, to obtain amorphous API by solidification of melt, the API has to be heated beyond its melting point, which may require expenditure of much energy, particularly when the API has a high melting point. Further, the high temperatures may chemically damage the API.

Another one of these processes, lyophilization, which is quite expensive process on large scale, and generally has limited capacity. Further, lyophilization with an organic solvent is often dangerous since it possesses a fire hazard.

Another one of these processes, evaporation of organic solvent at high temperature, which is also dangerous since it possesses a fire hazard.

Another one of these processes, spray drying, which consists of bringing together a highly dispersed liquid and a sufficient volume of hot air to produce evaporation and drying of the liquid droplets. Spray-drying however is often limited to aqueous solutions unless special expensive safety measures are taken. Also, in spite of the short contact time, certain undesirable physical and chemical characteristics of the emerging solids are in particular cases unavoidable. The turbulence present in a spray drier as a result of the moving air may alter the product in an undesirable manner.

Letermovir was approved for the treatment for prophylaxis of cytomegalovirus (CMV) infection and is currently marketed under the proprietary name Prevymis in the USA. The chemical name of Letermovir is (S)-{8-Fluoro-2-[4-(3-methoxyphenyl)-1-piperazinyl]-3-[2-methoxy-5-(trifluoromethyl) phenyl]-3,4-dihydro-4- quinazolinyl}acetic acid and the structure of Letermovir is as per Formula-I as depicted below.

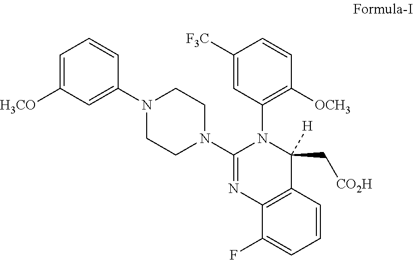 [SMILES: COc1cccc(N2CCN(C3=Nc4c(F)cccc4[C@H](CC(=O)O)N3c3cc(C(F)(F)F)ccc3OC)CC2)c1]

Letermovir is described in US 2005/0065160 for use in methods of prophylaxis or methods of treatment of viral diseases, HCMV infections. The synthesis of Letermovir is also disclosed in the examples 14 and 15 of US'160 and Letermovir is isolated using chromatography followed by acid/base precipitation process. However, US'160 is silent about polymorphic form and other physicochemical properties of the compound.

The synthesis of Letermovir is also disclosed in the example 11 of US 2009/0221822 and in this, Letermovir is isolated in amorphous solid by evaporation of ethanolic solution of the product up to dryness.

The synthesis of Letermovir is also disclosed in the example 6A of US 2015/0045371 and in this, Letermovir is precipitated in amorphous solid by adding water to ethanolic solution of the product.

The isolation of amorphous Letermovir is also disclosed in the examples of US 2016/0145216. The process involves hydrolysis reaction of Letermovir ester derivative to Letermovir at first place and then Letermovir was extracted in methyl tert-butyl ether (MTBE) solvent. The solvent was switched from MTBE to acetone/acetonitrile and finally the amorphous Letermovir was isolated by either use of i) a roller dryer, or ii) by precipitation of an acetonic or acetonitrile solution of Letermovir into an excess of stirred water.

US'216 also discloses that amorphous Letermovir can be also isolated by spray drying or evaporation of a solution in an organic solvent, but the obtained

Letermovir yields and/or purity were insufficient due to huge amount of residual solvent remaining in the amorphous API Letermovir.

US'216 further discloses that high boiling point solvents such as DMF, DMSO, NMP, etc. and highly toxic solvents such as glyme (1,2-dimethoxy ethane) are not suitable for precipitation/crystallization of amorphous Letermovir. It also discusses that the use of methanol or ethanol (alcohol) are not suitable for precipitation/crystallization of amorphous Letermovir as they yield re-esterification by-product impurities.

US'216 further discloses that THF solvent is not suitable for precipitation/crystallization of amorphous Letermovir as it yields an unknown impurity in about 0.35% and importantly THF as residual solvent amount of greater than 20,000 ppm which is not acceptable according to the ICH guideline.

US'216 further discloses that Methyl ethyl ketone (MEK) solvent is not suitable for precipitation/crystallization of amorphous Letermovir as it yields sticky precipitate and MEK as residual solvent amount of greater than 10,000 ppm which is not acceptable according to the ICH guideline.

These documents are incorporated herein by reference in entirety for all the purposes.

Further, attempts to crystallize Letermovir API as a stable crystalline polymorph have also failed to date and it looks like Letermovir predominantly exists in amorphous form. As discussed hereinabove, the amorphous form can be isolated by using techniques like precipitation, by adding solution of Letermovir in antisolvent or by spray drying or roller drying. When Letermovir is isolated in amorphous form by using different organic solvent systems, it has been observed that Letermovir shows tendency of trapping the organic solvents more than permissible limits of ICH guideline.

Hence, a need exists in the art for a process that allows for preparation of amorphous form of the API Letermovir on an industrial scale by sufficient yield, purity and have residual solvents in accordance with ICH guideline.

Surprisingly, the inventors of the present application have unexpectedly found a precipitation process for the isolation of amorphous Letermovir in a pure form as describe below in detail.

### OBJECT OF THE INVENTION

The present invention is related to a process of isolating amorphous Letermovir by using precipitation process which has residual solvents in in accordance with ICH guidelines. The present process makes amorphous Letermovir ready to be formulated in a solid pharmaceutical formulation for oral administration and for use in methods of prophylaxis or methods of treatment of viral diseases.

Specifically, the objective of present invention is summarized as follows:
(1) Objective of the present invention is related to amorphous Letermovir, contains less than 5000 ppm of MTBE as residual solvent.
(2) Second objective of the present invention is related to amorphous Letermovir, contains less than 5000 ppm of heptane as residual solvent.
(3) Third objective of the present invention is related to a precipitation process of amorphous Letermovir, contains residual solvents in accordance with ICH guidelines.
(4) Another objective of the present invention is related to a precipitation process for isolating amorphous Letermovir comprising:
   a) adding MTBE solution of Letermovir to heptane to precipitate amorphous Letermovir,
   b) isolating the amorphous Letermovir from step a) via filtration or centrifugation,
wherein the amorphous Letermovir contains residual solvents in accordance with ICH guidelines.
(5) Another objective of the present invention is related to a precipitation process for isolating amorphous Letermovir comprising:
   a) adding MTBE solution of Letermovir to heptane to precipitate amorphous Letermovir,
   b) isolating the amorphous Letermovir from step a) via filtration or centrifugation,
   c) drying the amorphous Letermovir,
wherein the amorphous Letermovir contains residual solvents in accordance with ICH guidelines.
(6) Another objective of the present invention is related to a precipitation process for isolating amorphous Letermovir comprising:
   a) adding MTBE solution of Letermovir to heptane at temperature less than 0 degree Celsius to precipitate amorphous Letermovir,
   b) isolating the amorphous Letermovir from step a) via filtration or centrifugation, wherein the amorphous Letermovir contains residual solvents in accordance with ICH guidelines.
(7) Another objective of the present invention is related to a precipitation process for isolating amorphous Letermovir comprising:
   a) adding MTBE solution of Letermovir to heptane at temperature less than 0 degree Celsius to precipitate amorphous Letermovir,
   b) isolating the amorphous Letermovir from step a) via filtration or centrifugation,
   c) drying the amorphous Letermovir, wherein the amorphous Letermovir contains residual solvents in accordance with ICH guidelines.

### BRIEF DESCRIPTION OF THE DRAWINGS

FIG. 1: PXRD pattern of amorphous Letermovir of example 1.

FIG. 2: PXRD pattern of amorphous Letermovir of example 2.

FIG. 3: PXRD pattern of amorphous Letermovir of example 12.

FIG. 4: Certification of analysis of amorphous Letermovir of example 13.

FIG. 5A & 5B: HPLC result of amorphous Letermovir of example 13.

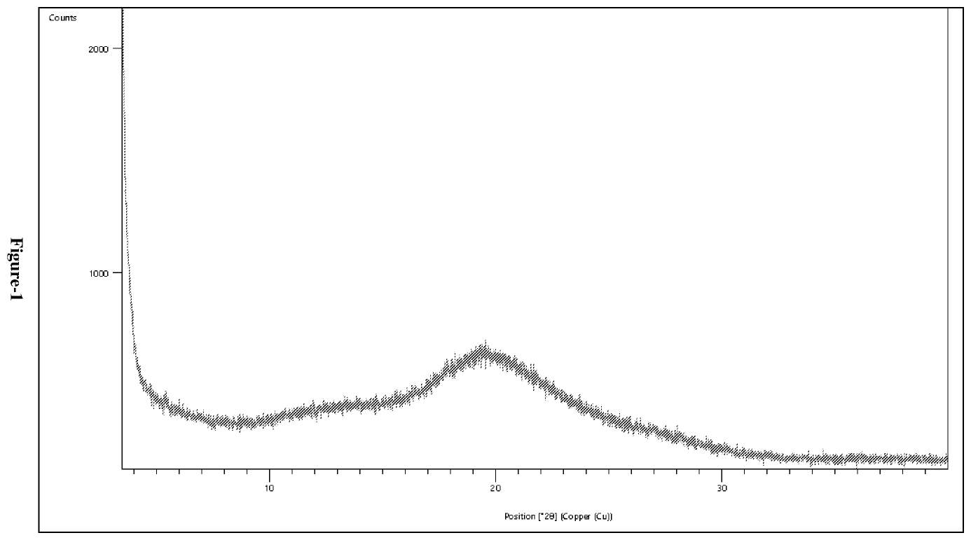

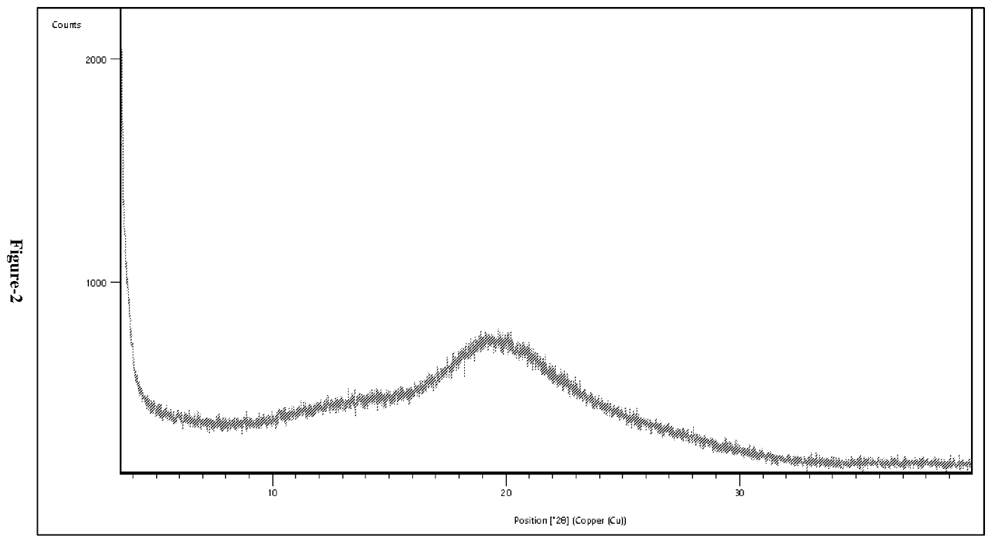

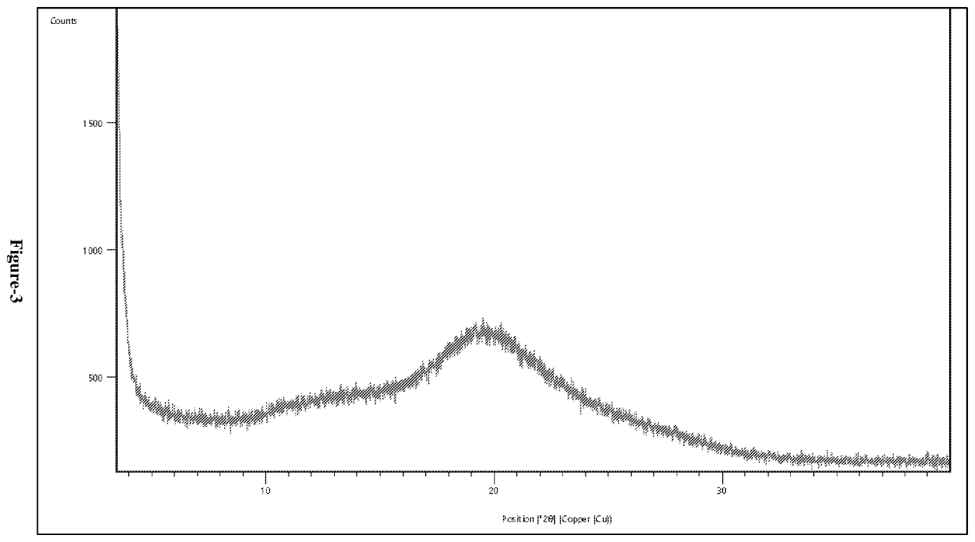

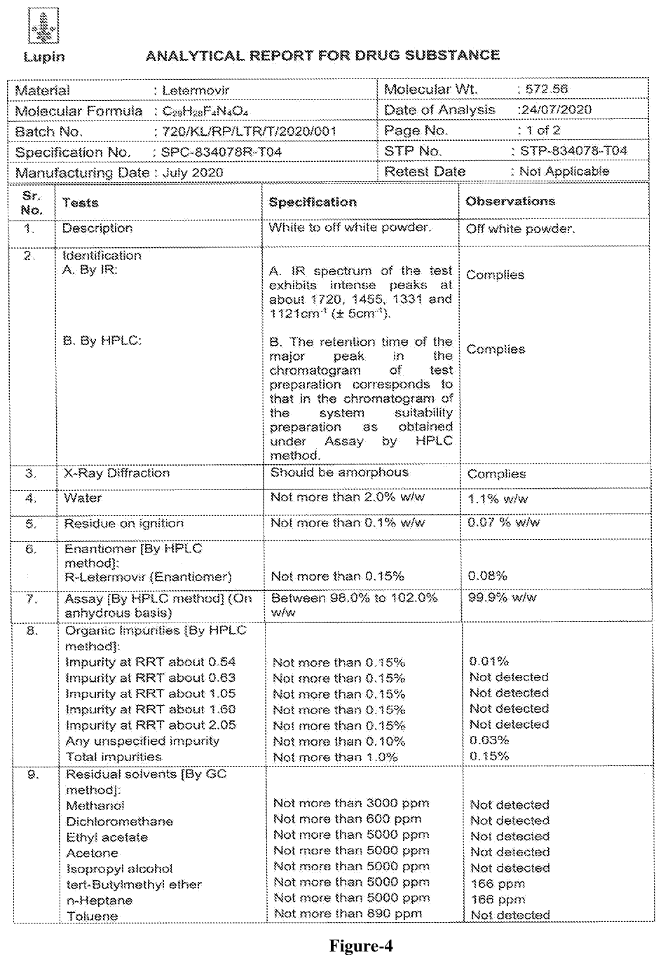

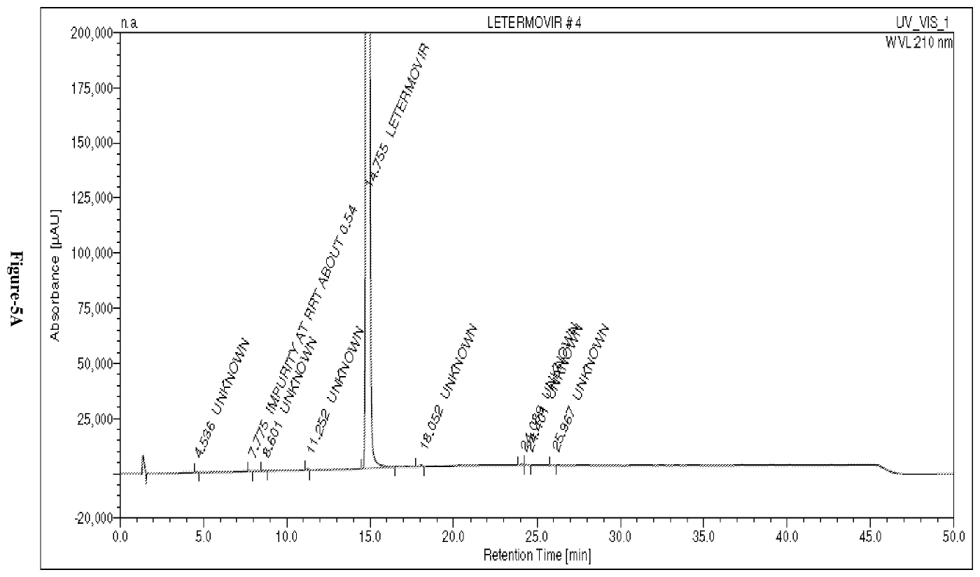

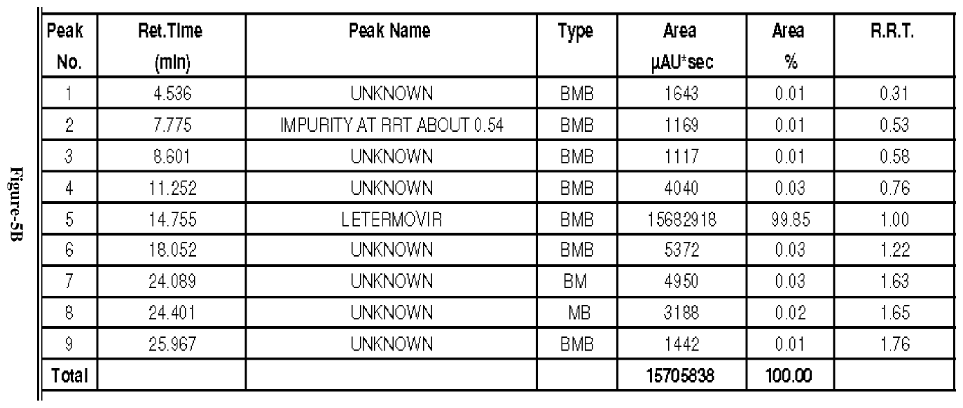

### DESCRIPTION OF THE INVENTION

The present invention relates to a process of isolating amorphous Letermovir by using precipitation process and which contains the residual solvents in accordance with ICH guidelines.

In one aspect, amorphous Letermovir is isolated by precipitation process from mixing of MTBE solution of Letermovir and heptane as anti-solvent. This precipitation solvent combination enables to produce amorphous Letermovir with the residual solvents in accordance with ICH guidelines.

In another aspect of the present invention, amorphous Letermovir is isolated by precipitation process from mixing of MTBE solution of Letermovir and heptane as anti-solvent at temperature less than 0 degree Celsius. According to the present invention, the precipitation temperature is about less than 0 degree Celsius, preferably at about 0 to −10 degree Celsius.

According to the present invention, the reaction mixture is stirred for enough time to complete the precipitation, preferably about 30 min. to 5 hours.

In another aspect of the present invention, the amorphous Letermovir produced by present invention, contains the residual solvents in accordance with ICH guidelines.

In another aspect of the present invention, the precipitation solvent(s) combination enables to produce amorphous Letermovir, contains residual solvents in accordance with ICH guidelines.

In another aspect of the present invention, the precipitated amorphous Letermovir is isolated via filtration or centrifugation.

In another aspect of the invention, the isolated amorphous Letermovir has a content of MTBE below 5000 ppm (pursuant to ICH guidelines), and a content of heptane below 5000 ppm (pursuant to ICH guidelines).

According to the present invention, the amorphous Letermovir is further dried under vacuum at about 40 degree Celsius to 100 degree Celsius for 2-24 hours, preferably at about 50 degree Celsius to 80 degree Celsius for 5-20 hours.

According to the present invention, the amorphous Letermovir, contains residual solvents in accordance with ICH guidelines as per below static headspace Gas chromatography method.
Weigh 100 mg of Letermovir sample and transfer to a 5 mL volumetric flask. Dissolve and dilute to the volume with 1-Methyl-2-pyrrolidinone. Pipette out 1.0 mL of this solution into a headspace vial. Seal with a septum and crimp cap. The analysis is performed by using Instrument: Perkin Elmer Gas Chromatograph; Column: DB-624 (30 meters length, 0.32 mm inner diameter, 1.8 μm Capillary Column); Detector: Flame Ionization Detector; Carrier gas: Nitrogen; Carrier gas Flow: 2.2 mL/min; Detector Temperature: 250° C.; Injector Temperature: 200° C.; Detector Range: 1; Attenuation: 32; Split: Split-less; Hydrogen Gas Flow: 45.0 mL/min.; Zero Air Gas Flow: 450.0 mL/min.; Equilibration Time: 0.5 min.; FID Filter Time Constant: 200 ms; Carrier Gas Flow Offset (Fixed): 3.0 mL/min.; Detector Offset: 5.0 mV; Injection volume calc. flow rate (HS): 2 ccm.
Oven Temp. Program:

| Ramp ° C./min | Temperature (° C.) | Hold (min) |
|---|---|---|
| — | 40 | 5 |
| 40 | 220 | 2 |

Head space parameters:
HS Mode: Constant; Oven Temperature: 90° C.; Needle Temperature: 100° C.; Transfer line temperature: 110° C.; GC Cycle time: 25 minutes; Thermostat time: 30 min.; Pressurization time: 3.0 min.; Injection Time: 0.05 min.; Injection volume: 0.1 mL; Withdrawal time: 0.1 min.; Headspace Pressure: 25 psig.

### Influence of Solvents on Isolation of Letermovir

The inventors of present application have found that the isolation solvent(s) play major role in isolation of amorphous Letermovir and thus specific and adequate solvent(s) is(are) needed to obtain Letermovir in a pure and chemically stable amorphous form.

During chemical development and optimization studies to obtain amorphous Letermovir in pharmaceutical grade, the following solvent combinations were investigated: MTBE/heptane, EA/heptane and isopropyl acetate/heptane.

The inventors of present application have found EA/heptane and isopropyl acetate/heptane solvent combinations to be not suitable as solvents for obtaining amorphous Letermovir in pharmaceutical grade either for quality reasons (residual solvents) or for the process of precipitation and precipitation itself.

The inventors have found alkyl acetate solvents (EA, isopropyl acetate) to be disadvantageous for isolating amorphous Letermovir in pharmaceutical grade since high amount of residual solvent has been trapped in the amorphous Letermovir.

Surprisingly, the inventors have found against expectation that MTBE/heptane solvent combination provides for sufficient precipitation properties to obtain Letermovir in an amorphous state and in pharmaceutical grade. Thus, MTBE/heptane solvent combination is the most preferred solvent combination to be applied for precipitation of amorphous Letermovir.

Furthermore, in accordance with the invention the residual solvents (MTBE, heptane) can effectively be removed in vacuo at elevated temperature (40-100 degree Celsius) without loss of purity or change of physicochemical properties in regard to the amorphous state.

The inventors have found that the amorphous Letermovir prepared as per present invention, has a content of MTBE less than 5000 ppm (pursuant to ICH guidelines), and/or a content of heptane less than 5000 ppm (pursuant to ICH guidelines).

In context with the stated above, particularly preferred subject matter of the present invention is provided by the following consecutively numbered and inter-related embodiments:

1. Letermovir according to Formula (I),

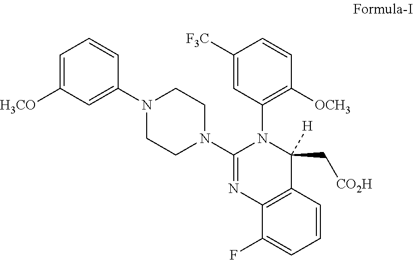 [SMILES: COc1cccc(N2CCN(C3=Nc4c(F)cccc4[C@H](CC(=O)O)N3c3cc(C(F)(F)F)ccc3OC)CC2)c1]

which is in the amorphous state and suitable for use in solid oral dosage forms, wherein said Letermovir is characterized by having residual solvents in accordance with ICH guidelines.

2. Letermovir according to embodiment 1, wherein said amorphous state is characterized by no detectable crystalline content/signal within the limit of detection of 2%, when said Letermovir is determined by any of the standard XRPD methods. One of the exemplary methods of analysis is described below, but not limited to
Powder sample of Letermovir is prepared on a rotating sample holder with an effective surface area of 10 mm (in diameter); powder diffraction patterns were recorded using a PANalytical Empyrean diffractometer equipped with pixel detector and Nickel filter using CuKα radiation operated at 45 kV and 40 mA. The measurement was performed using a step size of 0.007° with a step time of 24 s.

3. Letermovir according to any of the preceding embodiments, obtainable by the following process:
a) providing MTBE solution of Letermovir,
b) precipitating said amorphous Letermovir by mixing the MTBE solution with heptane as anti-solvent, and subsequently filtrating or centrifuging the amorphous Letermovir obtained.

4. Letermovir according to any of the preceding embodiments, obtainable by the following process:
a) providing MTBE solution of Letermovir,
b) precipitating said amorphous Letermovir by mixing the MTBE solution with heptane as anti-solvent at temperature less than 0 degree Celsius, and subsequently filtrating or centrifuging the amorphous Letermovir obtained.

5. Letermovir according to embodiments 3 and 4, wherein the process according to step b) has a final drying step.

6. Letermovir according to any of the preceding embodiments, obtainable by the following process:
a) adding MTBE solution of Letermovir to heptane to precipitate amorphous Letermovir,
b) isolating the amorphous Letermovir from step a) via filtration or centrifugation,
   wherein the amorphous Letermovir contains residual solvents in accordance with ICH guidelines.

7. Letermovir according to any of the preceding embodiments, obtainable by the following process:
a) adding MTBE solution of Letermovir to heptane at temperature less than 0 degree Celsius to precipitate amorphous Letermovir,
b) isolating the amorphous Letermovir from step a) via filtration or centrifugation,
   wherein the amorphous Letermovir contains residual solvents in accordance with ICH guidelines.

8. Letermovir according to embodiments 6 and 7, wherein the process according to step b) has a final drying step.

9. Letermovir according to any of the preceding embodiments, wherein said Letermovir in the amorphous state has a content of MTBE less than 5000 ppm and/or a content of heptane less than 5000 ppm, when said MTBE or heptane content is determined by the gas chromatography method.

10. Solid pharmaceutical formulation comprising the amorphous Letermovir, wherein said solid pharmaceutical formulation is orally administrable.

11. Solid pharmaceutical formulation according to embodiment 10, wherein the amorphous Letermovir is isolated according to the preceding embodiments and contains residual solvents according to embodiment 6.

12. Solid pharmaceutical formulation according to embodiment 11 for use in a method for prophylaxis or method of treatment for diseases associated with the group of Herpesviridae, preferably associated with cytomegalovirus (CMV), even more preferably associated with human cytomegalovirus (HCMV).

### DEFINITION

The term “metastable” with the context of amorphous Letermovir denotes a chemical state of temporary energy trap or a somewhat stable intermediate stage of a system the energy of which may be lost in discrete amounts.

The term “residual solvents” in terms of pharmaceuticals are defined here as organic volatile chemicals that are used or produced in the manufacture of drug substances or excipients, or in the preparation of drug products, as in the present case drugs substances based on Letermovir.

The term “amorphous” denotes the characteristic that no long-range order of neighboring molecular units is present or being a disorganized solid mass. Accordingly, the amorphous is exhibiting no detectable crystalline content/signal attributable when analyzed by an appropriate crystallographic method.

Accordingly, throughout the specification the expressions “amorphous, amorphous form, amorphous state” with the context of the present invention denotes material exhibiting no indication of crystallinity within the limit of detection of 2% by using standard PXRD methods and thus exhibits no detectable crystalline content/signal when analyzed by an appropriate crystallographic method. Typically Powder X-Ray Diffraction (PXRD) is used to determine the crystalline content of the material in accordance with the invention. One of the exemplary methods of analysis is described below, but not limited to:
The sample was prepared on a rotating sample holder with an effective surface area of 10 mm (in diameter). Powder diffraction patterns were recorded using a PANalytical Empyrean diffractometer equipped with pixel detector and Nickel filter using CuKα radiation operated at 45 kV and 40 mA. The measurement was performed using a step size of 0.007° with a step time of 24 s.

The term “ICH guideline(s)” within the scope of the invention denotes the International Conference on Harmonization of impurities: Guideline for residual solvents Q3C(R6). The objective of this guideline is to recommend acceptable amounts for residual solvents in pharmaceuticals for the safety of the patient. The guideline recommends use of less toxic solvents and describes levels considered to be toxicologically acceptable for some residual solvents. The guideline applies to all dosage forms and routes of administration. Higher levels of residual solvents may be acceptable in certain cases such as short term (30 days or less) or topical application.

The terms “pure/purified” in view of the API Letermovir characterizes the API in that it contains residual solvents in accordance with ICH guidelines in accordance with the instant invention.

The terms “pharmaceutical grade” in view of the API Letermovir characterizes any active or inactive drug, biologic, reagent, etc., manufactured under Good Manufacturing Practices (GMP) which is approved, conditionally approved, or indexed by the Food and Drug Administration (FDA) or for which a chemical purity standard has been written or established by a recognized compendia (e.g., United States Pharmacopeia-National Formulary (USP/NF) or British Pharmacopeia(BP)).

Further said terms denote that residual MTBE content does not exceed 5000 ppm and residual heptane content does not exceed 5000 ppm when determined by Gas Chromatography (GC).

### ABBREVIATIONS

Throughout the specification the following abbreviations apply:
“API” denotes active pharmaceutical ingredient
“MTBE” denotes Methyl tert-butyl ether, also known as methyl tertiary butyl ether, is an organic compound with molecular formula (CH₃)₃COCH₃. MTBE is a volatile, flammable, and colorless liquid that is not readily soluble in water.
“DMF” denotes dimethylformamide.
“EA” denotes ethyl acetate.
“THF” denotes tetrahydrofuran.
“DMSO” denotes dimethyl sulfoxide.
“NMP” denotes N-methyl-2-pyrrolidone.
“IPAc” denotes isopropyl acetate
“MEK” denotes methyl ethyl ketone.
“XRPD” denotes X Ray Powder Diffraction.
“ppm” denotes part per million.
“PXRD” denotes Powder X-Ray Diffraction.
“HPLC” denotes High Performance Liquid Chromatography.

Present invention is further illustrated with the following non-limiting examples.

### EXAMPLES

Example 1: To 600 ml heptane, solution of 20 gm Letermovir in 140 ml MTBE was added at −5° C. to −10° C. temperature. The reaction mixture was further maintained for 1 hour at same temperature. The precipitate was filtered under vacuum and dried at 55° C. to 65° C. temperature in vacuum to yield Letermovir as an amorphous state. (Yield 88%).

Example 2: Letermovir of Example 2 was prepared as same manner with same quantities as in Example 1 except using heptane 300 ml. (Yield 85%).

Example 3: To 150 ml heptane, solution of 15 gm Letermovir in 60 ml MTBE was added at 0° C. to −5° C. temperature. The reaction mixture was further maintained for 1 hour at same temperature. The precipitate was filtered under vacuum and dried at 55° C. to 65° C. temperature in vacuum to yield amorphous Letermovir. (Yield 87%)

Example 4: To 60 ml heptane, solution of 4 gm Letermovir in 20 ml MTBE was added at −5° C. to −10° C. temperature., The reaction mixture was further maintained for 1 hour at same temperature. The precipitate was filtered under vacuum and dried at 65° C. temperature in vacuum to yield amorphous Letermovir. (Yield 85%).

Example 5: To 30 ml heptane, solution of 2 gm Letermovir in 14 ml MTBE was added at −5° C. to −10° C. temperature. The reaction mixture was further maintained at same temperature for 1 hour. The precipitate was filtered under vacuum and dried at 65° C. temperature for 24 hours to yield Letermovir in amorphous state. (Yield 85%).

Example 6: Letermovir of Example 6 was prepared as same manner as in Example 5, except addition of Letermovir solution is performed at 40° C. to 45° C. temperature with maintaining at same temperature for 30 min. followed by maintaining at 20° C. temperature for 1 hour. (Yield 95%).

Example 7: To 60 ml heptane, solution of 2 gm Letermovir in 14 ml MTBE was added at −5° C. to −10° C. temperature. The reaction mixture was further maintained at same temperature for 1 hour. The precipitate was filtered under vacuum and dried at 65° C. temperature for 24 hours to yield Letermovir in amorphous state. (Yield 85%).

Example 8: Letermovir of Example 8 was prepared as same manner as in Example 7, except addition of the Letermovir solution is performed at 30° C. to 35° C. temperature and with maintaining the solution at same temperature for 1 hour. (Yield 90%).

Example 9: Letermovir of Example 9 was prepared as same manner as in Example 8 except using 30 ml of heptane (Yield 80%).

Example 10: To 60 ml heptane, solution of 2 gm Letermovir in 8 ml MTBE was added at 35° C. to 40° C. temperature. The reaction mixture was further maintained at same temperature for 1 hour. The precipitate was filtered under vacuum and dried at 65° C. temperature for 24 hours to yield amorphous Letermovir. (Yield 90%).

Example 11: Amorphous Letermovir of Example 11 was prepared as same manner as in Example 10 (Yield 90%).

Example 12: To 45 ml heptane, solution of 2 gm Letermovir in 10 ml MTBE was added at 15° C. to 20° C. temperature. The reaction mixture was further maintained at same temperature for 1 hour. The precipitate was filtered under vacuum and dried at 65° C. temperature for 24 hours to yield amorphous Letermovir. (Yield 85%).

Example 13: To 21.0 liters heptane, solution of 700 gm Letermovir in 4.9 liters MTBE was added at −5 ° C. to −10 ° C. temperature. The reaction mixture was further maintained for 1-2 hour at same temperature. The precipitate was filtered under vacuum and dried at 65° C. to 70° C. temperature in vacuum to yield Letermovir as an amorphous state. Purity by HPLC 99.85 (Yield 93%).

Example 14: To 20 ml heptane, solution of 1 gm Letermovir in 3 ml ethyl acetate was added at 30° C. to 35° C. temperature. The reaction mixture was further maintained at 25° C. temperature for 1 hour. The precipitate was filtered under vacuum and dried at 50° C. temperature for 13 hours to yield Letermovir. (Yield 90%).

Example 15: To 30 ml heptane, solution of 1 gm Letermovir in 3 ml isopropyl acetate was added at 30° C. to 35° C. temperature. The reaction mixture was further maintained at 20° C. temperature for 1 hour. The precipitate was filtered under vacuum and dried at 50° C. temperature for 13 hours to yield Letermovir. (Yield 90%).

Example 16: To 60 ml heptane, solution of 2 gm Letermovir in 14 ml isopropyl acetate was added at −5° C. to −10° C. temperature. The reaction mixture was further maintained at same temperature for 1 hour. The precipitate was filtered under vacuum and dried at 65° C. temperature to yield Letermovir. (Yield 92%)

Example 17: To 60 ml heptane, solution of 2 gm Letermovir in 14 ml Ethyl acetate was added at −5° C. to −10° C. temperature. The reaction mixture was further maintained at same temperature for 1 hour. The precipitate was filtered under vacuum and dried at 65° C. temperature to yield Letermovir. (Yield 92%)

Results of influence of temperature and solvent combination on quality of Letermovir:

| | Temperature | | | | |
|---|---|---|---|---|---|
| | condition | | Residual Solvents | | |
| Ex. | during | Solvent(s) for | in ppm | | |
| No. | precipitation | precipitation | MTBE | Heptane | PXRD |
| 1 | −5° C. | MTBE/heptane | 93 | 1283 | Amorphous |
| | to −10° C. | | | | |
| 2 | −5° C. | MTBE/heptane | 755 | 1087 | Amorphous |
| | to −10° C. | | | | |
| 3 | 0° C. | MTBE/heptane | 631 | 163 | Amorphous |
| | to −5° C. | | | | |
| 4 | −5° C. | MTBE/heptane | 424 | 234 | Amorphous |
| | to −10° C. | | | | |
| 5 | −5° C. | MTBE/heptane | 139 | 837 | Amorphous |
| | to −10° C. | | | | |
| 6 | 40° C. | MTBE/heptane | 27719 | 54270 | Not |
| | to 45° C. | | | | analyzed* |
| 7 | −5° C. | MTBE/heptane | 1336 | 923 | Amorphous |
| | to −10° C. | | | | |
| 8 | 30° C. | MTBE/heptane | 2521 | 17381 | Not |
| | to 35° C. | | | | analyzed* |
| 9 | 30° C. | MTBE/heptane | 320 | 18494 | Not |
| | to 35° C. | | | | analyzed* |
| 10 | 30° C. | MTBE/heptane | 1242 | 23344 | Amorphous |
| | to 35° C. | | | | |
| 11 | 30° C. | MTBE/heptane | 2087 | 33724 | Amorphous |
| | to 35° C. | | | | |
| 12 | 15° C. | MTBE/heptane | 326 | 8226 | Amorphous |
| | to 20° C. | | | | |
| 13 | 5° C. | MTBE/heptane | 166 | 166 | Amorphous |
| | to 10° C. | | | | |
| 14 | 30° C. | EA/heptane | 38902 ** | 8318 | Not |
| | to 35° C. | | (EA) | | analyzed* |
| 15 | 30° C. | IPAc/heptane | 137944 ** | 36 | Not |
| | to 35° C. | | (IPAc) | | analyzed* |
| 16 | −5° C. | IPAc/heptane | 7764 ** | 16135 | Not |
| | to −10° C. | | (IPAc) | | analyzed* |
| 17 | −5° C. | EA/heptane | 1662 ** | 21238 | Not |
| | to −10° C. | | (EA) | | analyzed* |

*= Failed in residual solvents as per ICH guidelines, hence the sample was not analyzed for PXRD.
** = Precipitation solvents as residual solvents reported in ppm in the example 14 to 17.

1. Letermovir according to Formula-I, 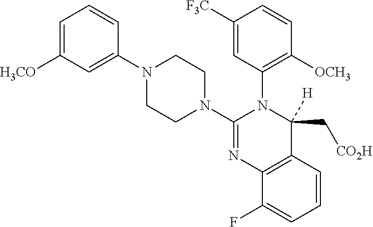 [SMILES: COc1cccc(N2CCN(C3=Nc4c(F)cccc4[C@H](CC(=O)O)N3c3cc(C(F)(F)F)ccc3OC)CC2)c1]
Formula-I which is in amorphous state, wherein said Letermovir is characterized by having residual solvents in accordance with ICH guidelines.

2. Letermovir according to claim 1, obtainable by the following process:
a) providing MTBE solution of Letermovir, b) precipitating said amorphous Letermovir by mixing the MTBE solution with heptane, and subsequently filtrating or centrifuging the amorphous Letermovir obtained.

3. Letermovir according to claim 1, obtainable by the following process:
a) providing MTBE solution of Letermovir, b) precipitating said amorphous Letermovir by mixing the MTBE solution with heptane as at temperature less than 0 degree Celsius, and subsequently filtrating or centrifuging the amorphous Letermovir obtained.

4. Letermovir according to claim 2, wherein the process according to step b) has a final drying step.

5. Letermovir according to claim 1, obtainable by the following process:
a) adding MTBE solution of Letermovir to heptane to precipitate amorphous Letermovir,
b) isolating the amorphous Letermovir from step a) via filtration or centrifugation,
wherein the amorphous Letermovir contains residual solvents in accordance with ICH guidelines.

6. Letermovir according to claim 1, obtainable by the following process:
a) adding MTBE solution of Letermovir to heptane at temperature less than 0 degree Celsius to precipitate amorphous Letermovir, b) isolating the amorphous Letermovir from step a) via filtration or centrifugation, wherein the amorphous Letermovir contains residual solvents in accordance with ICH guidelines.

7. Letermovir according to claim 5, wherein the process according to step b) has a final drying step.

8. Letermovir according to claim 1, wherein said Letermovir in amorphous state has a content of MTBE less than 5000 ppm and/or a content of heptane less than 5000 ppm, when said MTBE or heptane content is determined by the gas chromatography method.

9. Solid pharmaceutical formulation comprising the amorphous Letermovir, wherein said solid pharmaceutical formulation is orally administrable.

10. Solid pharmaceutical formulation according to claim 9, wherein the amorphous Letermovir is isolated and contains residual solvents according with ICH guidelines.

11. Solid pharmaceutical formulation according to claim 10 for use in a method for prophylaxis or method of treatment for diseases associated with the group of Herpesviridae, preferably associated with cytomegalovirus (CMV), even more preferably associated with human cytomegalovirus (HCMV).

12. Letermovir according to claim 3, wherein the process according to step b) has a final drying step.

13. Letermovir according to claim 6, wherein the process according to step b) has a final drying step.

14. Letermovir according to claim 2, wherein said Letermovir in amorphous state has a content of MTBE less than 5000 ppm and/or a content of heptane less than 5000 ppm, when said MTBE or heptane content is determined by the gas chromatography method.

15. Letermovir according to claim 3, wherein said Letermovir in amorphous state has a content of MTBE less than 5000 ppm and/or a content of heptane less than 5000 ppm, when said MTBE or heptane content is determined by the gas chromatography method.

16. Letermovir according to claim 4, wherein said Letermovir in amorphous state has a content of MTBE less than 5000 ppm and/or a content of heptane less than 5000 ppm, when said MTBE or heptane content is determined by the gas chromatography method.

17. Letermovir according to claim 5, wherein said Letermovir in amorphous state has a content of MTBE less than 5000 ppm and/or a content of heptane less than 5000 ppm, when said MTBE or heptane content is determined by the gas chromatography method.

18. Letermovir according to claim 6, wherein said Letermovir in amorphous state has a content of MTBE less than 5000 ppm and/or a content of heptane less than 5000 ppm, when said MTBE or heptane content is determined by the gas chromatography method.

19. Letermovir according to claim 7, wherein said Letermovir in amorphous state has a content of MTBE less than 5000 ppm and/or a content of heptane less than 5000 ppm, when said MTBE or heptane content is determined by the gas chromatography method.
